(19) United States
(12) Patent Application Publication
(10) Pub. No.: US 2016/0106679 A1
(43) Pub. Date: Apr. 21, 2016

(54) TABLET WITH INCREASED DRUG LOAD OF ODANACATIB

(71) Applicant: SANDOZ AG, Basel (CH)

(72) Inventors: Johannes Raneburger, Kundl (AT); Ludwig Englmeier, Holzkirchen (DE); Andreas Krekeler, Holzkirchen (DE); Michael Sedlmayr, Holzkirchen (DE)

(21) Appl. No.: 14/890,271

(22) PCT Filed: May 16, 2014
(86) PCT No.: PCT/EP2014/060164

§ 371 Date: Nov. 10, 2015

(30) Foreign Application Priority Data
May 16, 2013 (EP) 13168095.1

(51) Int. Cl. A61K 9/20; A61K 31/165

(52) U.S. Cl. CPC A61K 9/2054; A61K 31/165; A61K 9/2009; A61K 9/2095

(57) ABSTRACT

The present invention relates to pharmaceutical tablets comprising amorphous compound I in the form of its free base or pharmaceutically acceptable salts of compound I.

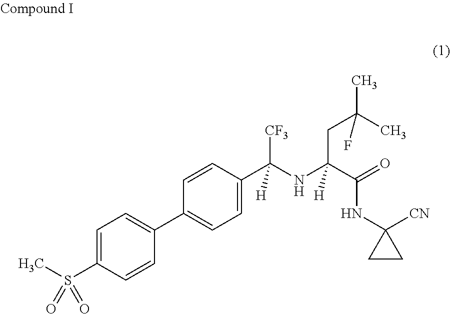 [SMILES: CC(C)(F)C[C@H](N[C@@H](c1ccc(-c2ccc(S(C)(=O)=O)cc2)cc1)C(F)(F)F)C(=O)NC1(C#N)CC1]

The present invention relates to pharmaceutical tablets comprising amorphous odanacatib or pharmaceutically acceptable salts thereof. Odanacatib is hereinafter also referred to as Compound I.

Compound I has the formula (1)

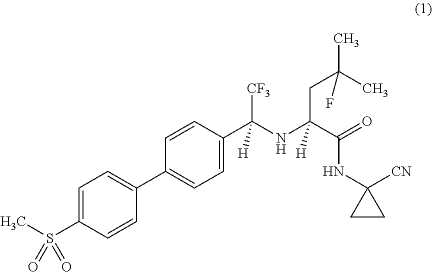 [SMILES: CC(C)(F)C[C@H](N[C@@H](c1ccc(-c2ccc(S(C)(=O)=O)cc2)cc1)C(F)(F)F)C(=O)NC1(C#N)CC1]

Compound I free base and acceptable salts thereof are disclosed in the Patent application WO 2003/075836 A2.

Compound I is an oral cathepsin-K inhibitor which is in clinical trials for the treatment of postmenopausal osteoporosis. Odanacatib is known to be highly crystalline with low aqueous solubility, and its oral bioavailability is highly dependable on vehicle, dosage and sample preparation (J. Y. Gauthier et al., Bioorg. Med. Chem. Lett. 18 (2008): 923-928).

In a published clinical trial patients treated once weekly with 20-50 mg of odanacatib showed increases in lumbar spine, hip and femoral neck BMD, while decreases in BMD at the same sites were seen in patients treated with low doses of odanacatib. Odanacatib is foreseen to be used in a dose of from 20 mg to 50 mg once weekly. Thus, a tablet with a dose of from 20-50 mg of odanacatib is desirable.

WO 2009/140105 A2 discloses pharmaceutical compositions based on solid dispersions comprising amorphous compound I. These formulations are prepared by spray drying or hot melt extrusion processes and they are prepared by combining 10-20% of compound I with 80-90% of a polymer, which polymer is needed to form an amorphous system with compound I. The tablets prepared from the amorphous compound I containing system have a final drug load of from 5.0% to 8.334% amorphous compound I of the total tablet formulation. A tablet with a unit dose of 50 mg and having a final drug load of 5.0% would, however, have a total mass of 1000 mg, which is not an optimal value from the perspective of patient compliance. However, increasing the drug load of an amorphous compound while at the same time retaining the beneficial dissolution properties of a tablet with a low drug load of the amorphous active pharmaceutical ingredient is not trivial for a compound with a high inherent tendency to crystallize, like odanacatib.

Despite the progress described in the art with regard to odanacatib oral pharmaceutical compositions there remains a need for improved oral pharmaceutical compositions of odanacatib, which can be taken easily and which can improve patient compliance. The oral pharmaceutical composition has to provide a plasma level of odanacatib which is sufficient for an effective therapy. This is dependent on the solubility and the release behavior of the solid form of odanacatib which is used in the pharmaceutical composition. Moreover, the dissolution behavior of the pharmaceutical composition and the chemical and mechanical stability of odanacatib when present in the context of the pharmaceutical composition is important. In the case of a tablet, such tablet should not be too large to allow a good swallowing. However, the dimensions of a tablet are dependent not only on the amount of odanacatib, but also on the amounts of excipients needed to keep odanacatib in a desired state and/or chemically stable. Moreover, type and amount of the excipients used in combination with the process for preparation of the pharmaceutical dosage form are important for release properties, bioavailability, stability and the ease with which an industrial manufacturing process for the pharmaceutical composition can be implemented.

The present inventors have noted difficulties in the production of the tablets comprising amorphous compound I as described in WO 2009/140105 A2. Tablets with an increased load of odanacatib compared to those described in WO 2009/140105 A2—prepared by simply increasing the amount of odanacatib relative to the other excipients, in particular the HPMC-AS—have unfavorable dissolution characteristics. Further, in the tablets described in WO 2009/140105 A2 flexibility with regard to the quantities of excipients, e.g. disintegrants, is limited due to the high load of the tablets with the polymer in order to keep the odanacatib amorphous. Thus, there still is a need for commercially acceptable odanacatib dosage forms for oral administration with good patient convenience and acceptance.

Moreover, the spray dried amorphous odanacatib with HPMC-AS shows difficulties with regard to pulversization after spray-drying—with the intermediate after the spray drying step being somewhat tacky—so that a sufficiently small particle size distribution of the spray-dried amorphous material and a homogeneous distribution of the amorphous material in the final tablet is difficult to achieve.

In accordance with the present invention, it has now unexpectedly been found that stable and convenient galenic tablets comprising amorphous odanacatib in a higher amount as previously described are obtainable. The present inventors have found that pharmaceutically acceptable oral solid dosage forms in the form of tablets, being particularly convenient to administer and wherein odanacatib is in its amorphous state, may be obtained by preparation of tablets by compression methods. More specifically, the tablets of the invention may be prepared by milling and dry granulation, followed by compression methods.

It is a characteristic of the tablet according to the invention that it contains a relatively high content of amorphous odanacatib given the relatively small amount of excipients. This enables the production of physically small tablets wherein odanacatib is in its amorphous state and even stays so upon storage.

Tablets according to the invention surprisingly provide for the administration of amorphous odanacatib in tablets having a smaller size than was hitherto possible for a given unit dose of odanacatib. The tablets of the invention are, despite the increased drug loading, small, and, therefore, convenient to administer. This leads to a better patient compliance.

The present invention relates to a tablet comprising a pharmacologically effective amount of amorphous Compound I present in an amount of from 10% to 25%, e.g. at least from 11, 12, 13, 14 or 15% to 20, 21, 22, 23 or 24%. In particular, the amount of Compound I may vary from 11 to 23%, e.g. from 12 to 20% in weight based on the total weight of the tablet. Thus, the tablet of the invention has an increased load of amorphous Compound I, which is beneficial for patient compliance. The tablet of the invention is also smaller and requires smaller quantities of excipients, thus rendering them more environmentally friendly and avoiding excessive amounts of excipients.

The term “tablet” as used herein is intended to comprise compressed pharmaceutical oral dosage forms of all shapes and sizes, whether coated or uncoated.

The term “amorphous” as used herein means a solid state of a body which does not have a long-range crystalline order as detectable by XRD (X-ray diffraction).

The term absence of crystalline odanacatib free base in a sample can be determined by the absence of a peak at any one of the prominent peak positions+/−0.1° 2Theta for crystalline odanacatib free base in an XRD. Prominent peaks in the XRD spectrum of crystalline odanacatib free base, which can be used for this purpose, are at positions 7.81°, 15.09°, 15.48°, 17.64°, 17.95°, 20.11°, 21.15° and 23.62° (measurement is done at a wavelength of 0.15419 nm, using Cu Kα_{1,2} radiation. Of course, the skilled person will have prepared a corresponding reference sample having the same constituents as the to-be-tested sample, but not containing odanacatib, in order to assure that any peak at the above-given 2Theta positions will be specific for crystalline odanacatib, i.e. not attributable to one of the other sample components.

The term “inert matrix” as used herein means an inorganic inert material having a BET specific surface area of at least 1 m²/g. In the context of the present invention, the BET specific surface area is determined using the Brunauer-Emmet-Teller method described in “The Journal of the American Chemical Society”, Vol. 60, page 309, February 1938 and corresponding to the International Standard ISO 5794/1 (Appendix D). The inert matrix is preferably inherently amorphous.

Compound I may be in the free base form or pharmaceutically acceptable salts thereof. The calculation of the active moiety corresponds to Compound I in the free base form. It is preferred that Compound I is in the form of the free base.

The present invention also relates to a tablet comprising
(a) a pharmacologically effective amount of amorphous Compound I or pharmaceutically acceptable salt thereof,
(b) a pharmaceutically acceptable inert matrix,
(c) a modified cellulose, and
(d) at least one further excipient,
   wherein the amount of Compound I or the pharmaceutically acceptable salt thereof, calculated as the percentage of the content in weight of the active moiety based on the total the tablet, is from about 10% to 25%, e.g. at least from 11, 12, 13, 14 or 15% to 20, 21, 22, 23 or 24%. In particular the amount of Compound I may vary from 11 to 23%, e.g. from 12 to 20% in weight of the active moiety based on the total weight of the tablet.

Pharmaceutically acceptable salts of compound I are, for example, salts with inorganic acids, such as hydrochloric acid, hydrobromic acid, sulfuric acid, phosphoric acid, methanesulfuric acid, trifluoromethanesulfuric acid, benzenesulfuric acid and p-toluenesulfuric acid, and salts with organic acids, such as acetic acid, malic acid, tartaric acid, citric acid, lactic acid, oxalic acid, succinic acid, fumaric acid, maleic acid, benzoic acid, alicylic acid and mandelic acid, to name but a few. It is however preferred that Compound I is in the form of its free base.

The pharmaceutically acceptable inert matrix (b) is an inert material with a sufficiently high BET specific surface area of at least 1 m²/g which allows some adsorption of amorphous odanacatib to it. Preferably, the BET specific surface area is at least 10 m²/g, such as from 10 m²/g to 1000 m²/g, for example from 20 m²/g to 500 m²/g. Examples can be a phosphate or a silicate, provided that they have a desired BET specific surface area. A preferred inert matrix is an amorphous magnesium aluminosilicate such as amorphous magnesium aluminometasilicate. A synthetic version of amorphous aluminometasilicate is, for example, commercially available from Fuji Chemical Industry Co. Ltd. Examples for phosphates with a surface area of at least 1 m²/g are calcium phosphates, e.g. di- or tribasic calcium phosphate.

The modified cellulose (c) is preferably selected from the group consisting of alkylcellulose, e.g. methylcellulose, ethylcellulose, propylcellulose; hydroxalkylcellulose, e.g. hydroxymethylcellulose, hydroxyethylcellulose, hydroxypropylcellulose; hydroxyalkylalkylcellulose, e.g. hydroxyethylmethylcellulose (HEMC), hydroxypropylmethylcellulose (HPMC); carboxyalkylcellulose, e.g carboxymethylcellulose (CMC), carboxymethylhydroxyethylcellulose (CMHEC), hydroxyethylcarboxymethylcellulose (HECMC), sodium carboxymethylcellulose, cellulose acetate phthalate (CAP), hydroxypropylmethylcellulose acetate (HPMCA), hydroxypropylmethylcellulose phthalate (HPMCP), hydroxypropylmethylcellulose acetate succinate (HPMCAS) or mixtures thereof.

Even more preferably, the chemically modified cellulose and/or cellulose ether is selected from the group consisting of alkylcellulose, e.g. methylcellulose, ethylcellulose, propylcellulose; hydroxalkylcellulose, e.g. hydroxymethylcellulose, hydroxyethylcellulose, hydroxypropylcellulose; hydroxyalkylalkylcellulose, e.g. hydroxyethylmethylcellulose (HEMC), hydroxypropylmethylcellulose (HPMC); carboxyalkylcellulose, e.g carboxymethylcellulose (CMC), carboxymethylhydroxyethylcellulose (CMHEC), hydroxyethylcarboxymethylcellulose (HECMC), sodium carboxymethylcellulose, or mixtures thereof. A particularly preferred chemically modified cellulose is hydroxypropylmethylcellulose, preferably hydroxypropylmethylcellulose which is further functionalized with a carboxylic acid, such as hydroxypropylmethylcellulose acetate phthalate, such as HPMCAS-HF, HPMCAS-MF or HPMCAS-LF. HPMCAS-HF has an acetyl content of 10.2-14.0%, a succinoyl content of 4.0-8.0%, a methoxyl content of 22.0-26.0% and a hydroxypropyl content of 6.0-10.0%, with a average particle size of not more than 10 μm. HPMCAS-MF has an acetyl content of 7.0-11.0%, a succinoyl content of 10.0-14.0%, a methoxyl content of 21.0-25.0% and a hydroxypropyl content of 5.0-9.0%, with an average particle size of not more than 10 μm. HPMCAS-LF has an acetyl content of 5.0-9.0%, a succinoyl content of 14.0-18.0%, a methoxyl content of 20.0-24.0% and a hydroxypropyl content of 5.0-9.0%, with an average particle size of not more than 10 μm.

A further excipient can be selected from diluents, glidants, lubricants, surfactants and disintegrants. Reference is made to the extensive literature on the subject for these and other excipients and procedures mentioned herein, see in particular Handbook of Pharmaceutical Excipients, Third Edition, edited by Arthur H. Kibbe, American Pharmaceutical Association, Washington, USA and Pharmaceutical Press, London; and Lexikon der Hilfsstoffe für Pharmazie, Kosmetik and angrenzende Gebiete edited by H. P. Fiedler, 4th Edition, Edito Cantor, Aulendorf and earlier editions which are incorporated herein by reference.

In a preferred embodiment the tablet of the present invention comprises as further excipients a diluent, a disintegrant and a lubricant, preferably further comprising a glidant, even more preferably further comprising a glidant and a surfactant.

Examples of diluents are microcrystalline cellulose, calcium phosphate, calcium carbonate, starch, spray-dried lactose, anhydrous lactose, lactose monohydrate and mannitol. Spray dried lactose is a preferred diluent.

Examples of disintegrants include but are not restricted to maize starch; CMC-Ca; CMC-Na; microcrystalline cellulose; cross-linked PVP, e.g. as known and commercially available under the trade names Crospovidone®, Polyplasdone®, available commercially from the ISP company, or Kollidon® XL; alginic acid; sodium alginate; and guar gum. Cross-linked PVP, e.g. Crospovidone is a preferred disintegrant.

Examples of glidants include one or more of the following: silica, colloidal silica, e.g. colloidal silica anhydrous, e.g. Aerosil® 200, magnesium trisilicat, powdered cellulose, starch and talc. Colloidal silica anhydrous or/and colloidal silicon dioxide are preferred glidants.

Examples of lubricants include one or more of the following: Mg-, Al- or Ca-stearate, PEG 4000-8000 and/or talc. Magnesium stearate is a preferred lubricant.

Examples of surfactants include one or more of the following: an alkyl sulfate salt, such as sodium laurylsulfate, or a poloxamer. Sodium lauryl sulfate is a preferred surfactant.

It will be appreciated that any given excipient may serve more than one function e.g. as disintegrant, binder, glidant, and/or lubricant.

In a preferred embodiment the invention relates to a tablet of the present invention which comprises as further excipients a diluent, a disintegrant and a lubricant, preferably further comprising a glidant, even more preferably further comprising a glidant and a surfactant, wherein diluent, disintegrant, lubricant, glidant and surfactant are the preferred diluent, disintegrant, lubricant, glidant and surfactant as described in the preceeding paragraphs.

Even more preferably, the diluent, disintegrant, lubricant, glidant and surfactant are the preferred diluent, disintegrant, lubricant, glidant and surfactant as described in the preceeding paragraphs, the modified cellulose (c) is hydroxypropylmethylcellulose which is further functionalized with a carboxylic acid, such as hydroxypropylmethylcellulose acetate phthalate, such as HPMCAS-HF, HPMCAS-MF or HPMCAS-LF,

and the pharmaceutically acceptable inert matrix (b) is a phosphate or a silicate, in particular an amorphous magnesium aluminosilicate such as amorphous magnesium aluminometasilicate.

In a preferred aspect of the invention, the tablet comprises the at least one further excipient (d) in a total amount of about 40% to 60% in weight based on the total weight of the tablet. When more than one further excipient is present, it is preferred that the diluent is present in an amount of from 70% to 90% by weight based on the total weight of all further excipients (d).

According to the present invention, amorphous Compound I is present in an amount of from 10% to 25% in the tablets of the present invention, which is a fairly high drug load for amorphous odanacatib. Thus, in the tablets of the present invention odanacatib is preferably present in an amount of from 18 mg to 52 mg amorphous odanacatib. Examples are tablets containing 20 mg odanacatib or 50 mg odanacatib.

The tablets of the present invention have an advantage of allowing a higher drug load of amorphous odanacatib. As a consequence, the total mass of the tablet can be kept within acceptable limits which are suitable for easy administration to the patient, even if a pharmaceutically effective dose of amorphous odanacatib is to be administered. Thus the tablets of the present invention preferably have a total weight of from 80 mg to 500 mg. Preferably the tablets of the present invention have a total weight of from 80 mg to 200 mg when amorphous odanacatib free base is present in an amount of about 20 mg. Alternatively, the tablets of the present invention have a total weight of from 200 mg to 500 mg when amorphous odanacatib free base is present in an amount of about 50 mg.

According to the invention, the process for the preparation of the tablets comprises mixing compound I, or a pharmaceutically acceptable salt thereof, with a pharmaceutically acceptable inert matrix, such as a phosphate or a silicate, and a modified cellulose, such as HPMCAS, milling the mixture, e.g. until a purely amorphous mixture is obtained, mixing the purely amorphous mixture with at least one further excipient and compressing the obtained mixture to obtain the tablet.

For example odanacatib free base can be mixed with amorphous magnesium aluminosilicate and hydroxypropylmethylcellulose acetate succinate, milled in an oscillating ball mill, such as a Retsch mill, until a purely amorphous powder, for example as judged by the absence of any prominent peak indicative of remaining crystalline material in an XRPD, is obtained. The obtained amorphous mixture can then be dry granulated together with, for example, a diluent, such as spray dried lactose, a disintegrant, such as crosscarmellose, a gildant, such as fumed silica, a lubricant, such as magnesium stearate, and a surfactant, such as sodium lauryl sulfate, for example via dry granulation by roller compaction. After compaction the dry granulate can be milled and the milled granulation compacted, optionally after blending with a further portion of lubricant.

Without wishing to be bound by any theory—the milled inert matrix together with the milled modified cellulose provide a sufficiently large inner surface which helps to assure good dissolution properties of the tablet of the present invention.

The present invention also relates to a process for the preparation of a tablet of the invention, which process comprises
(i) dissolving odanacatib, preferably odanacatib free base, and a modified cellulose in a solvent selected from methanol, ethanol, n-propanol, isopropanol, a C3-C5 ketone/C1-C5 alcohol mixture and a C3-C5 nitrile/C1-C5 alcohol mixture;
(ii) bringing the solution into contact with an inert inorganic matrix material and removing the solvent from the mixture to form an amorphous powder;
(iii) mixing the amorphous powder with pharmaceutically acceptable excipients to form a mixture; and
(iv) compressing the mixture obtained in step (iii) to form a tablet.

Preferred solvents for step (i) are methanol, ethanol, an aceton/methanol mixture, an aceton/ethanol mixture, an aceton/n-propanol mixture and an aceton/isopropanol mixture. In step (ii) it is preferred that a solution of odanacatib, in particular of odanacatib free base, and the modified cellulose, in particular HPMC-AS, is prepared and then brought into contact with the inert inorganic carrier, preferably amorphous magnesium aluminometasilicate, followed by removal of the solvent. This is preferably effected by spray-drying. Spray-drying is well known to the person skilled in the art and suitable spray-drying techniques are described, for example, in Remington's Pharmaceutical Sciences, 20^{th} edition, Mack Publishing Co., 2000.

Thus, in a preferred mode, a solution of odanacatib free base and HPMS-AS is sprayed onto solid amorphous magnesium aluminometasilicate in a spray-drying apparatus. This leads to rapid removal of the solvent due to the typical temperature and pressure conditions in a spray-drying apparatus.

Alternatively, odanacatib, in particular of odanacatib free base, the modified cellulose, in particular HPMC-AS, and the inert inorganic carrier, preferably amorphous magnesium aluminometasilicate, are brought into contact in the solvent. A suspension will form, where odanacatib and the modified cellulose are dissolved, but the inert carrier remains undissolved, from which the solvent is removed. Solvent removal is preferably effected by spray-drying, e.g. by spraying the suspension onto the inert surface of a spray drying apparatus.

Further mixing with excipients and compression into tablets are routine measures well known to the skilled person.

Optionally, the tablets may be coated, preferably as described herein after.

According to the invention, the coating process may be performed at low temperature, e.g. between 30 and 40° C., preferably between 32 and 39° C., most preferably at a temperature ranging from around 35 to around 38° C. The coating process may be performed with a spray rate preferably in the range of 30 to 105 g of coating dispersion per kg of cores per hour, preferably of 35 to 105 g. It has surprisingly been found that neither swelling of the disintegrants, e.g. Crospovidone®, nor sticking of the cores occurred during spraying of the coating mixture, as it would be expected by the person skilled in the art by processing at low temperatures.

Moreover, the coated tablets exhibit improved abrasion resistance. The physical and chemical stability may be tested in conventional manner, e.g. the tablets may be tested as such by measurement of dissolution, friability, disintegration time, assay for Compound I degradation products, appearance and/or microscopy, e.g. after storage at room temperature, i.e. 25° C., and/or storage at 40° C.

By “total weight of the tablet” is meant the weight of an uncoated tablet, e.g. a tablet core. The tablet cores may vary in shape and be, for example, round, oval, oblong, cylindrical or any other suitable shape. A characteristic of tablets according to the invention is their small size having regard to the amount of amorphous Compound I or amorphous Compound I salt contained therein.

In a preferred embodiment of the invention tablets obtained by the compression method described above are round or oval. The edges of the tablets may be beveled or rounded. Most preferably, the tablets are ovaloid and/or round. The tablets according to the invention may be scored. The ovaloid tablet may be small in dimension e.g. 10 to 20 mm in length, preferably 15 to 20 mm, most preferably 17 to 19 mm; 5 to 10 mm in width, preferably 6.5 to 8 mm. The thickness of the tablet is from 4 to 8 mm, preferably 6 to 8 mm. Compression forces of between 10 to 20 kN are used to prepare the compressed tablet, preferably, 12 to 18 kN. Preferably, the ovaloid tablet contains 50 mg of Compound I. The round tablet may be of the following dimensions, e.g. 5 to 15 mm in diameter, preferably 7 to 10 mm, most preferably about 9 mm. The thickness of the tablet may be from 2 to 5 mm, preferably 2.5 to 4 mm. Compression forces of between 6 to 18 kN are used to prepare the compressed tablet, preferably, 8 to 14 kN. Preferably, the round tablet contains 50 mg of Compound I. The tablets of the invention comprising about 20 mg of Compound I may furthermore have a hardness of from about 30 to 140 N, e.g. 40 to 140 N, 30 to 100 N, 40 to 100 N, preferably 50 to 80 N. The tablets of the invention comprising about 50 mg of Compound I may have a hardness of 100 to 270 N, e.g. 100 to 250 N, 130 to 200 N.

The disintegration time of the tablet may be of about 20 min or less. Preferably, for the 20 mg Compound I tablet, the disintegration time is ranging from about 2 to 10 min, preferably 4 to 10 min, e.g. 4 to 8 min. For the 50 mg Compound I tablet, the disintegration time is, preferably ranging from about 7 to 15 min, preferably 8 to 15 min, e.g. 8 to 14 min.

The friability of the tablets is measured according to the US Pharmacopeia. The friability of the tablets according to the invention monitored following the recommendation of the US Phramacopeia is 0%.

The tablets of the invention may furthermore be colored and/or the tablets or coating marked so as to impart an individual appearance and to make them instantly recognizable. The use of dyes can serve to enhance the appearance as well as to identify the tablets. Dyes suitable for use in pharmacy typically include carotinoids, iron oxides or chlorophyll. The tablets of the invention may be marked using an imprint code.

Procedures which may be used may be conventional or known in the art or based on such procedures e.g those described in L. Lachman et al. The Theory and Practice of Industrial Pharmacy, 3rd Ed, 1986, H. Sucker et al, Pharmazeutische Technologie, Thieme, 1991, Hagers Handbuch der pharmazeutischen Praxis, 4th Ed. (Springer Verlag, 1971) and Remington's Pharmaceutical Sciences, 13th Ed., (Mack Publ., Co., 1970) or later editions.

The tablets of the invention are useful for the human indication of Compound I, e.g. treatment of postmenopausal osteoporosis, as indicated by standard tests. The activity and characteristics of the tablets of the invention may be indicated in standard clinical trials and/or animal trials.

The tablets of the invention comprising a pharmacologically effective amount of Compound I or Compound I salt may be administered as the sole active drug or with another active drug may be envisaged, e.g. together with simultaneous or separate administration of other drugs.

The present invention also related to the tablets of the present invention which have been packaged in convenient packaging material. Some preferred packaging materials are polyethylene, polypropylene, polyethylene terephthalate, polystyrene, polyvinyl chloride and polyvinylidene chloride. Other preferred packaging materials are polypropylene foil and polyvinyl chloride foil. Another suitable form of packaging is an alu-alu blister.

Preferred tablets for packaging are tablets of the present invention which comprise as further excipients a diluent, a disintegrant and a lubricant, preferably further comprising a glidant, even more preferably further comprising a glidant and a surfactant, wherein diluent, disintegrant, lubricant, glidant and surfactant are the preferred diluent, disintegrant, lubricant, glidant and surfactant as described in the preceeding paragraphs. Even more preferred tablets for packaging are tablets of the present invention wherein the diluent, disintegrant, lubricant, glidant and surfactant are the preferred diluent, disintegrant, lubricant, glidant and surfactant as described in the preceeding paragraphs,

the modified cellulose (c) is hydroxypropylmethylcellulose which is further functionalized with a carboxylic acid, such as hydroxypropylmethylcellulose acetate phthalate, such as HPMCAS-HF, HPMCAS-MF or HPMCAS-LF,
and the pharmaceutically acceptable inert matrix (b) is a phosphate or a silicate, in particular an amorphous magnesium aluminosilicate such as amorphous magnesium aluminometasilicate.

The present invention also relates to a pharmaceutical blister pack comprising tablets of the present invention. Preferably the blister pack comprises a packaging material such as polyethylene, polypropylene, polyethylene terephthalate, polystyrene, polyvinyl chloride, aluminum and polyvinylidene chloride.

Preferred tablets for packaging in the pharmaceutical blister pack of the invention are tablets of the present invention which comprise as further excipients a diluent, a disintegrant and a lubricant, preferably further comprising a glidant, even more preferably further comprising a glidant and a surfactant, wherein diluent, disintegrant, lubricant, glidant and surfactant are the preferred diluent, disintegrant, lubricant, glidant and surfactant as described in the preceeding paragraphs. Even more preferred tablets for packaging in the pharmaceutical blister pack of the invention are tablets of the present invention wherein the diluent, disintegrant, lubricant, glidant and surfactant are the preferred diluent, disintegrant, lubricant, glidant and surfactant as described in the preceeding paragraphs,

the modified cellulose (c) is hydroxypropylmethylcellulose which is further functionalized with a carboxylic acid, such as hydroxypropylmethylcellulose acetate phthalate, such as HPMCAS-HF, HPMCAS-MF or HPMCAS-LF,
and the pharmaceutically acceptable inert matrix (b) is a phosphate or a silicate, in particular an amorphous magnesium aluminosilicate such as amorphous magnesium aluminometasilicate.

A preferred blister pack comprising tablets of the present invention comprises at least two tablets of the present invention and six tablets without odanacatib. Preferably the blister pack also contains instructions to the patient that the odanacatib-containing tablets be taken once weekly while one tablet be taken per day. The odanacatib-free tablets act as filler tablets which are to be taken between the once weekly odanacatib-containing tablets. This helps to get the patient used to taking one tablet per day, while at the same time taking only one odanacatib-containing tablet per week, thus establishing a robust once-weekly dosage regimen of odanacatib for the patient. Examples of such blister packs of the present invention are blisters containing three odanacatib-containing tablets and at least twelve odanacatib-free dummy tablets or blisters containing four odanacatib-containing tablets and at least eighteen odanacatib-free dummy tablets or blisters containing five odanacatib-containing tablets and at least 24 odanacatib-free dummy tablets or blisters containing six odanacatib-containing tablets and at least 30 odanacatib-free dummy tablets. The blister packs of the present invention help to improve patient compliance for a once weekly dosage regimen of odanacatib.

Preferred odanacatib-containing tablets for packaging in the preferred pharmaceutical blister pack of the invention are tablets of the present invention which comprise as further excipients a diluent, a disintegrant and a lubricant, preferably further comprising a glidant, even more preferably further comprising a glidant and a surfactant, wherein diluent, disintegrant, lubricant, glidant and surfactant are the preferred diluent, disintegrant, lubricant, glidant and surfactant as described in the preceeding paragraphs. Even more preferred tablets for packaging in the pharmaceutical blister pack of the invention are tablets of the present invention wherein the diluent, disintegrant, lubricant, glidant and surfactant are the preferred diluent, disintegrant, lubricant, glidant and surfactant as described in the preceeding paragraphs,

the modified cellulose (c) is hydroxypropylmethylcellulose which is further functionalized with a carboxylic acid, such as hydroxypropylmethylcellulose acetate phthalate, such as HPMCAS-HF, HPMCAS-MF or HPMCAS-LF,
and the pharmaceutically acceptable inert matrix (b) is a phosphate or a silicate, in particular an amorphous magnesium aluminosilicate such as amorphous magnesium aluminometasilicate.

The present invention also relates to a tablet comprising
(a) a pharmacologically effective amount of amorphous Compound I or pharmaceutically acceptable salt thereof,
(b) a pharmaceutically acceptable inert matrix, such as a phosphate or a silicate, in particular an amorphous magnesium aluminosilicate such as amorphous magnesium aluminometasilicate,
(c) a cyclodextrine, such as a beta-cyclodextrine, for example hydroxypropyl beta cyclodextrine or sulfobutylether beta cyclodextrine, and
(d) at least one further excipient,
   wherein the amount of Compound I or the pharmaceutically acceptable salt thereof, calculated as the percentage of the content in weight of the active moiety based on the total the tablet, is from about 10% to 25%, e.g. at least from 11, 12, 13, 14 or 15% to 20, 21, 22, 23 or 24%. In particular the amount of Compound I may vary from 11 to 23%, e.g. from 12 to 20% in weight of the active moiety based on the total weight of the tablet (cyclodextrine embodiment).

Preferred tablets of this cyclodextrine embodiment are tablets which comprise as further excipients a diluent, a disintegrant and a lubricant, preferably further comprising a glidant, even more preferably further comprising a glidant and a surfactant, wherein diluent, disintegrant, lubricant, glidant and surfactant are the preferred diluent, disintegrant, lubricant, glidant and surfactant as described in the preceeding paragraphs. Even more preferred tablets of this cyclodextrine embodiment are tablets wherein the diluent, disintegrant, lubricant, glidant and surfactant are the preferred diluent, disintegrant, lubricant, glidant and surfactant as described in the preceeding paragraphs.

The present invention also relates to a tablet comprising
(a) a pharmacologically effective amount of amorphous Compound I or pharmaceutically acceptable salt thereof,
(b) a pharmaceutically acceptable inert matrix, such as a phosphate or a silicate, in particular an amorphous magnesium aluminosilicate such as amorphous magnesium aluminometasilicate,
(c) a water soluble casein salts, such as sodium caseinate or potassium caseinate, and
(d) at least one further excipient,
   wherein the amount of Compound I or the pharmaceutically acceptable salt thereof, calculated as the percentage of the content in weight of the active moiety based on the total the tablet, is from about 10% to 25%, e.g. at least from 11, 12, 13, 14 or 15% to 20, 21, 22, 23 or 24%. In particular the amount of Compound I may vary from 11 to 23%, e.g. from 12 to 20% in weight of the active moiety based on the total weight of the tablet (casein embodiment).

Preferred tablets of this casein embodiment are tablets which comprise as further excipients a diluent, a disintegrant and a lubricant, preferably further comprising a glidant, even more preferably further comprising a glidant and a surfactant, wherein diluent, disintegrant, lubricant, glidant and surfactant are the preferred diluent, disintegrant, lubricant, glidant and surfactant as described in the preceeding paragraphs. Even more preferred tablets of this casein embodiment are tablets wherein the diluent, disintegrant, lubricant, glidant and surfactant are the preferred diluent, disintegrant, lubricant, glidant and surfactant as described in the preceeding paragraphs.

The present invention also relates to a tablet comprising
(a) a pharmacologically effective amount of amorphous Compound I or pharmaceutically acceptable salt thereof,
(b) a pharmaceutically acceptable inert matrix, such as a phosphate or a silicate, in particular an amorphous magnesium aluminosilicate such as amorphous magnesium aluminometasilicate,
(c) a polyvinyl caprolactam-polyvinyl acetate-polyethylene glycol graft copolymer, and
(d) at least one further excipient,
   wherein the amount of Compound I or the pharmaceutically acceptable salt thereof, calculated as the percentage of the content in weight of the active moiety based on the total the tablet, is from about 10% to 25%, e.g. at least from 11, 12, 13, 14 or 15% to 20, 21, 22, 23 or 24%. In particular the amount of Compound I may vary from 11 to 23%, e.g. from 12 to 20% in weight of the active moiety based on the total weight of the tablet (caprolactam embodiment).

Preferred tablets of this caprolactam embodiment are tablets which comprise as further excipients a diluent, a disintegrant and a lubricant, preferably further comprising a glidant, even more preferably further comprising a glidant and a surfactant, wherein diluent, disintegrant, lubricant, glidant and surfactant are the preferred diluent, disintegrant, lubricant, glidant and surfactant as described in the preceeding paragraphs. Even more preferred tablets of this caprolactam embodiment are tablets wherein the diluent, disintegrant, lubricant, glidant and surfactant are the preferred diluent, disintegrant, lubricant, glidant and surfactant as described in the preceeding paragraphs.

The following non-limitative examples illustrate the invention.

### EXAMPLE 1

### Tablet Formulation (50 mg Tablet)

### Composition Per Dosage Form Unit and Quantity Per Batch

| | | Composition | Quantity |
|---|---|---|---|
| Component | | per unit (mg) | per batch (g) |
| Compound I | | 50.00 | 20 |
| Hydroxypropyl methylcellulose - | (1.1) | 50.00 | 20 |
| AS-HF | | | |
| Neusilin ® | (1.2) | 50.00 | 20 |
| Croscarmellose sodium | (2.1) | 20.30 | 8.1 |
| Spray dried Lactose | (2.2) | 153.00 | 61.2 |
| Magnesium stearate | (2.3) | 4.22 | 1.7 |
| Sodiumlaurylsulfate | (2.4) | 6.75 | 2.7 |
| Synthetic amorphous | (2.5) | 3.37 | 1.35 |
| pyrogenic silica | | | |
| Total weight | | 337.64 | 135.05 |
| Units/batch | | | 400 |

Tablets of 50 mg of Compound I free base and of the above tablet are prepared by milling of a mixture of Compound I with (1.1) and (1.2) to form a purely amorphous mixture as determined by XRPD, blending with (2.1), (2.2), ⅖^{th} of (2.3), (2.4) and (2.5), dry granulating, milling, blending with the remaining (2.3), and compressing the resultant blend to form tablet cores.

Amorphous odanacatib is present in the obtained tablets and amorphous odanacatib remains present upon storage, which is surprising given the relatively high load of about 15% odanacatib in the obtained tablets.

### EXAMPLE 2

Tablets are prepared as in example 1, but replacing Hydroxypropyl methylcellulose-AS-HF with the same mass of Hydroxypropyl methylcellulose-AS-MF.

### EXAMPLE 3

Tablets containing odanacatib free base were prepared. A mixture of 3.0 g odanacatib free base with 3.0 g Hydroxypropyl methylcellulose-AS-HF and 3.0 g Neusilin® was milled for 15 min in a ball mill (Retsch mixer mill MM301) in a 50 ml grinding beaker at a frequency of 20 Hz to form a purely amorphous mixture as determined by XRPD. This mixture was then blended with 0.96 g croscarmellose sodium, 4.83 g spray dried lactose, 4.83 g microcrystalline cellulose and 0.19 g synthetic amorphous pyrogenic silica for 5 min (mixer Turbula T10B), then 0.19 g magnesium stearate was added and blending was continued for 2 min. The resulting blend was compressed using a compression force of 8 kN to form tablet cores containing 50 mg odanacatib free base each.

Odanacatib free base was present in its amorphous form in the obtained tablets, which is surprising given the relatively high load of about 15% odanacatib in the obtained tablets, and remains present upon storage.

### EXAMPLE 4

The dissolution of the tablets from example 3 was analyzed by a dissolution assay. Briefly, a tablet was put into a dissolution vessel, 900 ml 0.1M aqueous HCl solution containing 4% SDS were added (temperature 37° C.), and dissolution was effected by stirring with a paddle at 100 rpm. Odanacatib dissolved quickly, with more than 70% released within the first 30 min

1. A tablet comprising a pharmacologically effective amount of amorphous odanacatib free base of 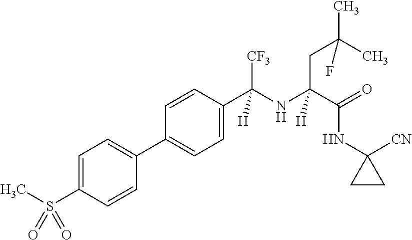 [SMILES: CC(C)(F)C[C@H](N[C@@H](c1ccc(-c2ccc(S(C)(=O)=O)cc2)cc1)C(F)(F)F)C(=O)NC1(C#N)CC1]
Compound 1 in an amount from 10% to 25% in weight of active moiety based on a total weight of the tablet.

2. The tablet of claim 1, further comprising
a pharmaceutically acceptable silicate;
a modified cellulose; and
at least one further excipient.

3. The tablet according to claim 1, wherein amorphous odanacatib is present in an amount of from 18 mg to 52 mg.

4. The tablet according to claim 1, wherein the total weight of the tablet is from 80 mg to 500 mg.

5. (canceled)

6. The tablet according to claim 2, wherein the pharmaceutically acceptable silicate is an amorphous magnesium aluminosilicate and/or wherein the modified cellulose is a hydroxypropylmethylcellulose functionalized with a carboxylic acid.

7. The tablet according to claim 2, wherein the at least one further excipient is present in an amount of from 40% to 60% by weight based on the total weight of the tablet.

8. The tablet according to claim 7, wherein a diluent constitutes 70% to 90% by weight based on the total weight of all of the at least one further excipients.

9. The tablet according to claim 7, wherein the least one further excipient comprises at least one further excipient selected from a binder, a glidant, a lubricant, a surfactant and a disintegrant.

10. The tablet according to claim 9, wherein the lubricant comprises magnesium stearate.

11. The tablet according to claim 9, wherein the disintegrant comprises crosscarmellose sodium.

12. The container suitable for packaging of pharmaceutical compositions, said container comprising a tablet according to claim 1.

13. The container according to claim 12, which container is a pharmaceutical blister pack comprising at least two tablets according to claim 1 and six tablets without odanacatib.

14. A process for the preparation of a tablet according to claim 1, which process comprises:
(i) mixing odanacatib, or pharmaceutically acceptable salts thereof, and an inert inorganic matrix material and a modified cellulose to form a first mixture;
(ii) milling the first mixture to form a milled first mixture;
(iii) mixing the milled first mixture with pharmaceutically acceptable excipients to form a second mixture; and
(iv) compressing the second mixture obtained in step (iii) to form a tablet.

15. A process for the preparation of a tablet according to claim 1, which process comprises:
(i) dissolving odanacatib and a modified cellulose in a solvent selected from methanol, ethanol, n-propanol, isopropanol, a C3-C5 ketone/C1-C5 alcohol mixture and a C3-C5 nitrile/C1-C5 alcohol mixture to form a solution;
(ii) bringing the solution into contact with an inert inorganic matrix material and removing the solvent from the mixture to form an amorphous powder;
(iii) mixing the amorphous powder with pharmaceutically acceptable excipients to form a mixture; and
(iv) compressing the mixture obtained in step (iii) to form a tablet.

16. The process according to claim 14, wherein the mixture from step (iii) is further dry granulated before compression to form a tablet.

17. The tablet according to claim 6, wherein the hydroxypropylmethylcellulose functionalized with a carboxylic acid is hydroxypropylmethylcellulose acetate succinate.
